(19) United States
(12) Patent Application Publication
(10) Pub. No.: US 2024/0366361 A1
(43) Pub. Date: Nov. 7, 2024

(54) BLOOD VESSEL COVER

(71) Applicant: Akeo HAGIWARA, Otsu-shi, Shiga (JP)

(72) Inventors: Akeo HAGIWARA, Otsu-shi (JP); Yosaku HAGIWARA, Osaka-shi (JP)

(73) Assignee: Akeo HAGIWARA, Otsu-shi, Shiga (JP)

(21) Appl. No.: 18/689,100

(22) PCT Filed: Aug. 22, 2022
(86) PCT No.: PCT/JP2022/031605

§ 371 Date: Mar. 5, 2024

(30) Foreign Application Priority Data
Sep. 8, 2021 (JP) 2021-146521

(51) Int. Cl. A61F 2/06; A61B 17/11

(52) U.S. Cl. CPC A61F 2/06; A61B 2017/1107

(57) ABSTRACT

A cylindrical vascular cover (10) is continuous around the entire circumference and placed outside a vein (4) anastomosed to an artery (3) or an artificial vessel, has a first part (11) from a midpoint (10c) to a first end (10a) and a second part (12) from the midpoint (10c) to a second end (10b) in an axial direction x of the vascular cover (10), and a diameter (d2) of a second virtual cylinder (T2) having a central axis parallel to a central axis (C) of the vascular cover (10) and inscribed in an inwall of the second part (12) is larger than a diameter (d1) of a first virtual cylinder (T1) having a central axis parallel to the central axis (C) and inscribed in an inwall of the first part (11).

### TECHNICAL FIELD

The present invention relates to a blood vessel cover used for an anastomosis site where blood vessels are anastomosed, and for example, a blood vessel cover that can be placed on an outer circumference of a vein at an anastomosis site where an artery or an artificial vessel that is anastomosed to an artery is anastomosed to the vein to form a shunt construction.

### BACKGROUND ART

For patients with serious kidney diseases including renal failure, hemodialysis treatment is regularly performed, in which blood is taken from the patient's body, waste products, excess water and minerals are removed with a dialyzer, and then the blood is returned to the patient's body. When hemodialysis is performed, a special needle is usually inserted into a vein. At this time, since the blood flow in the vein is not sufficient to carry out dialysis, the vein is anastomosed to an artery. Such a vessel is called a shunt, and the shunt is usually formed by making an incision in the skin of the arm to expose the artery and vein, making a small incision in the artery to which the vein is anastomosed, and diverting some of the blood flow from the artery to the vein. The vein may be directly anastomosed to artery, or an artificial blood vessel may be placed between the artery and the vein by anastomosing one end of the artificial blood vessel to the small incision of the artery and anastomosing the other end of the artificial blood vessel to the vein.

At a shunt construction, since there is a significant difference in elasticity between the artery and vein, when beating blood flows with high blood pressure in the artery flow into the vein that has markedly high extensibility at low pressure and has low elasticity at high pressure, blood turbulence and stress change to the vein wall occur. This results in intimal thickening of the anastomosis site and outflow tract vein, which can easily lead to pathological changes such as stenosis, occlusion, and thrombus formation. Failure to regulate shunt blood flow conditions at a high burden to the organism can lead to more extensive local pathologies (e.g., varicose formation or stenosis of downstream veins, Steel's syndrome due to excessive shunt blood flow) or systemic pathologies (e.g., heart failure due to markedly increased venous annular flow).

If conditions are favorable, appropriate remodeling may occur as a protective and adaptive response of the body, e.g., by elastic changes in the venous wall, and stenosis or obstruction due to intimal thickening may be avoided, or the shunt blood flow may be self-regulated to a state that is not burdensome to the body. However, if local conditions such as shunt blood flow and anastomotic geometry, or systemic conditions (e.g., diabetes, hypertension, arteriosclerosis, or blood conditions) are poor, it exceeds the extent to which an appropriate protective and adaptive response occurs to become a pathological biological response, leading to local or systemic disorder.

To address these issues, vascular banding is used, for example, in Non-patent document 1, where the vein is reinforced from the outside to prevent excessive blood pressure and consequent hyperextension and blood turbulence in the inner veins, in order to control the rapid increase in blood flow immediately after and during the early phase of the procedure. Patent document 1 discloses a covering for reinforcing natural veins for use as surgical implants, which is a mesh fabric net covering made by forming a knitted fabric that is seamless, tubular, and substantially pile-less. Patent documents 2 and 3 disclose that arteriovenous grafts (AVG) wrapped by a constrictive fiber matrix of biodegradable polymers show a throbbing radial deviation similar to the carotid artery.

### RELATED ART DOCUMENTS

### Patent Documents

Patent Document 1: JP 2004-535896 T
Patent Document 2: JP 2010-516437 T
Patent Document 3: JP 2013-509258 T

### Non-Patent Document

Non-patent document 1: “I Blood Access Problems associated with blood flow failure” by Hiroaki Haruguchi, Nihon Toseki Igakkai Zasshi Vol. 15, No. 1, 68-70, 2000

### SUMMARY OF THE INVENTION

### Problems to be Solved by the Invention

However, the above-described vascular banding could not sufficiently prevent lesions such as intimal thickening. In the conventional vascular banding, the reinforced vein wall is modified (arterialized) into an arterial wall-like structure only under certain conditions, but the blood pressure and pulsation are not buffered and delivered downstream as blood flows from the reinforced site to the non-reinforced vein, which is not a fundamental solution to the cause of intimal thickening. To solve this, the blood pressure and pulsation should be gradually reduced to be remodeled to a state where only low pressure without pulsatility is applied.

The present invention was made in view of the above circumstances, and the objective thereof is to provide a blood vessel cover that can reduce or prevent intimal thickening by remodeling the vein into a buffer system vessel that can deliver blood to downstream vein while gradually reducing blood pressure, pulse pressure, and blood flow through the lumen.

### Means for Solving the Problems

A blood vessel cover in accordance with an embodiment of the present invention that can solve the above problem is as follows.

[1] A cylindrical blood vessel that is continuous around the entire circumference and to be placed on an outer circumference of a vein that is anastomosed to an artery or to an artificial vessel, comprising:
a first end and a second end in an axial direction of the blood vessel cover; and
a first part from the first end to a midpoint between the first end and the second end, and a second part from the midpoint to the second end, wherein
a diameter of a second virtual cylinder having a central axis parallel to a central axis of the blood vessel cover and inscribed in an inner wall of the second part is larger than a diameter of a first virtual cylinder having a central axis parallel to a central axis of the blood vessel cover and inscribed in an inner wall of the first part.

The above blood vessel cover has the configuration where the minimum inner diameter in the second part is larger than the minimum inner diameter in the first part. Placing the first part of the blood vessel cover having such a configuration on the upstream side of the vein at the shunt construction allows the downstream side of the vein to be loosely covered, and allows the vein at the shunt construction to be remodeled so that it becomes a buffer vessel where the pulsatile blood flow with high arterial pressure that flows into the vein at the shunt construction can be gradually buffered towards downstream and finally shifted to venous blood flow. In addition, while veins may gradually grow outward in the process of remodeling into buffered vessels, the blood vessel cover with the above configuration does not interfere with this growth, and thus, can maintain a wide lumen of the covered blood vessel to ensure sufficient blood flow. Thus, the blood vessel of the present invention enables shunt construction that allows sufficient blood flow while reducing lesions such as intimal thickening by remodeling the vein into a buffer system vessel.

The blood vessel cover in accordance with embodiments of the present invention preferably includes the following [2] to [7].

[2] The blood vessel cover according to [1], comprising:
a first end part that is from the first end to a midpoint of the first part of the blood vessel cover in the axial direction, and that has a first inner diameter;
a middle part that is located on the second end side to the first end part, and that has a second inner diameter of 1.2 times or more the first inner diameter;
a first transition part with a progressively increasing inner diameter between the first end part and the middle part; and
a second transition part with a progressively increasing inner diameter between the middle part and the second end, wherein
the second end has a third inner diameter of 1.2 times or more the second inner diameter.

Placing the first end part of the blood vessel cover having such a configuration on the upstream side of the vein at the shunt construction allows the vein to be more loosely covered as it is more downstream, facilitating remodeling of the vein into a buffer system vessel and ensuring blood flow. In addition, the inner diameter of the second end is larger than the inner diameter of the middle part of the blood vessel cover, which can mitigate the effect of the sudden release of the restraint by the covering at the second end of the blood vessel cover.

[3] The blood vessel cover according to [2], wherein, in a cross-section in the axial direction, a boundary between the first end part and the first transition part, a boundary between the first transition part and the middle part, and a boundary between the middle part and the second transition part are curved.

This allows the vessel to be covered with the blood vessel cover with a smooth luminal wall, making the remodeling into a buffer system vessel easier.

[4] The blood vessel cover according to any one of [1] to [3], wherein a force required to expand the inner wall of the second part by 1.5 times in a radial direction from its natural state is less than a force required to expand the inner wall of the first part by 1.5 times in the radial direction from its natural state.

This facilitates remodeling of the vein into a buffer system vessel, because placing the first part of the blood vessel cover on the upstream side of the vein at the shunt construction allows the downstream side of the vein to be more loosely covered.

[5] The blood vessel cover according to any one of [1] to [4], having a length in the axial direction of 5 mm or longer.

Covering the vein at the shunt construction with the blood vessel cover having a length longer than a predetermined length facilitates remodeling of the vein into a buffer system vessel.

[6] The blood vessel cover according to any one of [1] to [5], comprising at least one of knitted fabric, woven fabric, and nonwoven fabric as a partial or whole component.

[7] The blood vessel cover according to any one of [1] to [6], having a bellows structure with periodically repeating peaks and valleys in the axial direction, wherein, in the axial direction, the blood vessel cover has a first end part from the first end to a midpoint of the first part, and a distance between adjacent peaks in the second part is longer than a distance between adjacent peaks in the first part.

This facilitates remodeling of the vein into a buffer system vessel, because placing the first end part of the blood vessel cover on the upstream side of the vein at the shunt construction allows the downstream side of the vein to be more loosely covered.

### Effects of the Invention

Covering the vein at the shunt construction by the blood vessel cover of the present invention having the above configuration can suppress incompatibility of vessel wall elasticity, turbulent blood flow, and excessive high flow rate to reduce or prevent intimal thickening by gradually changing the wall structure of the vein from the anastomosis site to the downstream to gradually change shear stress, pressure orthogonal to the vascular wall, blood flow, flow velocity, and range of change with beating in the interior of the covered vein. The reason why the blood vessel cover of the present invention achieves these effects may be due to the following.

Both arteries and veins consist of an intima, a tunica media, and an adventitia, and the arteries have the tunica media consisting of smooth muscle cell-rich smooth muscle layer and an elastic fiber layer including collagen fibers. The arteries have thick smooth muscle and elastic fiber layers so that pulsation change in the vessel wall is little to prevent turbulence generation and fluctuations in abrasion stress, even under the pressure of pulsating luminal blood flow. On the other hand, the veins have thin vessel walls and do not have the thick smooth muscle and elastic fiber layers as the arteries do. When arterial blood flows directly into such veins via arteriovenous shunts, lesions such as intimal thickening occur due to the substantial difference in elasticity between arteries and veins as described above. To prevent this, while the most upstream part of the vein at a shunt construction, i.e., the anastomosis site, is subjected to 100% pulsatile arterial pressure, the vein at the shunt construction needs to be remodeled into a buffer system vessel, which gradually decreases the blood pressure, pulsatility, blood flow, and maximum flow velocity towards the downstream of the vein to make the most downstream vein have low non-pulsatile pressure.

The blood vessel cover of the present invention, having the above configuration, can make the vein at the shunt construction remodeled into a buffer system vessel so that the pulsatile blood flow with high arterial pressure entering the veins at arteriovenous or artificial vessel-vein shunt construction can be gradually buffered downstream and finally shifted to venous blood flow, which is a buffer system vessel. As a result, blood turbulence and pulsatile change in the venous wall can be suppressed, preventing lesions such as intimal thickening.

In addition, the blood vessel cover of the present invention, having the above configuration, does not interfere with the gradual outward growth of veins in the process of remodeling them into low-pressure buffer system vessels, and as a result, the lumen of the vessel can be maintained wide enough to ensure sufficient blood flow. This enables shunt construction that ensure sufficient blood flow while suppressing lesions such as intimal thickening.

### BRIEF DESCRIPTION OF THE DRAWINGS

FIG. 1 is a schematic view of one example of shunt construction.

FIG. 2 is a schematic view of another example of shunt construction.

FIG. 3 is a perspective view of a blood vessel cover in accordance with one embodiment of the present invention.

FIG. 4 is a perspective view of a blood vessel cover in accordance with another embodiment of the present invention.

FIG. 5 is a perspective view of a blood vessel cover in accordance with still another embodiment of the present invention.

FIG. 6 is a perspective view of a blood vessel cover in accordance with still another embodiment of the present invention.

FIG. 7 is a perspective view of a blood vessel cover in accordance with still another embodiment of the present invention.

### MODE FOR CARRYING OUT THE INVENTION

Hereinafter, the present invention will be described based on the following embodiments, however, the present invention is not limited by the following embodiments and can be altered in design within a scope in compliance with the intent described above and below, and all the changes are to be encompassed within a technical scope of the present invention. Note that, in each drawing, hatching, reference signs for components, and the like may be omitted for convenience of description, and in such a case, the specification and other drawings are to be referred to. Furthermore, since the dimensions of the various components in the drawings are provided for the purpose of facilitating the understanding of the feature of the present invention, the dimensions may differ from the actual dimensions in some cases.

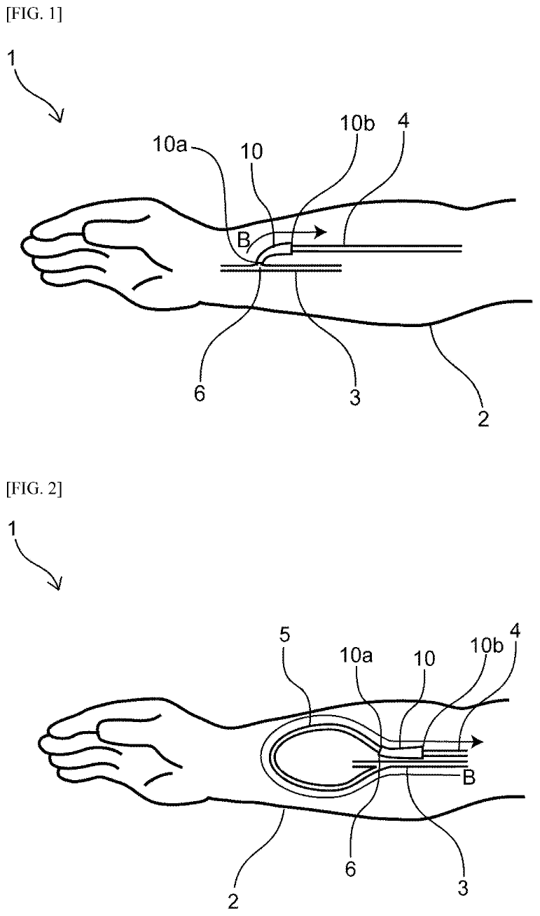

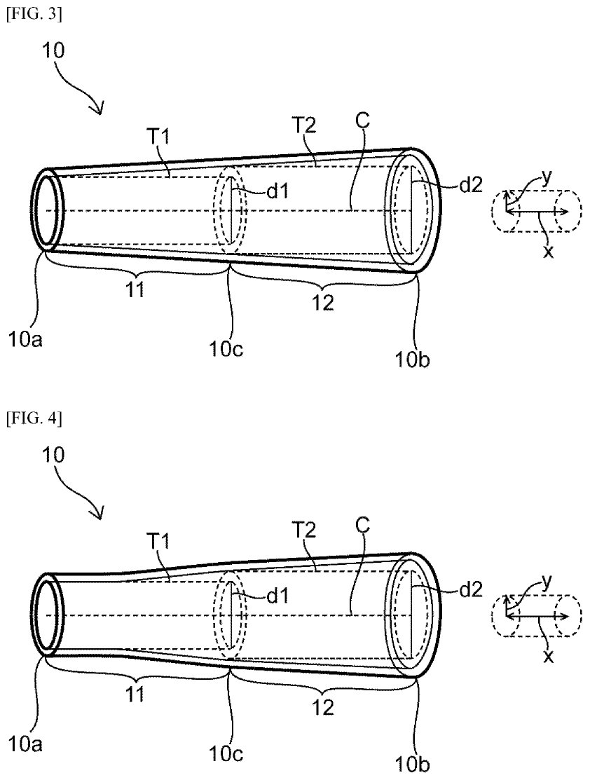

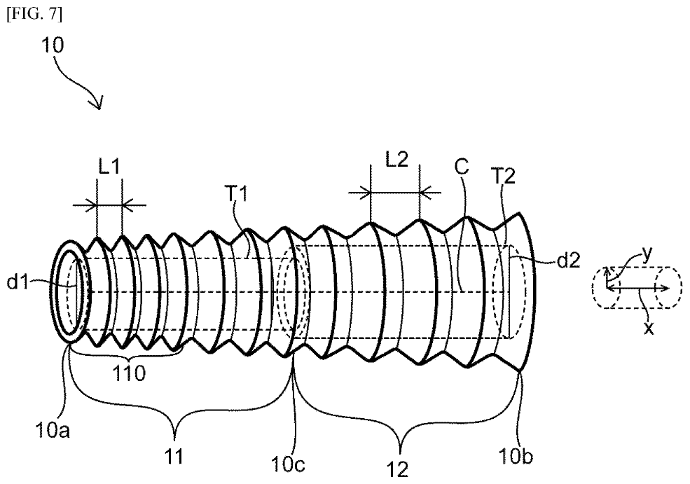

A blood vessel cover in accordance with embodiments of the present invention will be described referring to the figures. Note that the present invention is not limited to the embodiments shown in the figures. FIG. 1 is a schematic view in which an autologous vein is anastomosed to a small incision in an artery at a shunt construction, and FIG. 2 is a schematic view in which one end of an artificial vessel is anastomosed to a small incision in an artery and the other end of the artificial vessel anastomosed to a vein. FIG. 3 to FIG. 5 are perspective views of a blood vessel cover in accordance with different embodiments, respectively. FIG. 6 and FIG. 7 are perspective views of a blood vessel cover in accordance with still different embodiments, respectively.

A shunt construction 1 can be formed by performing an arteriovenous anastomosis as shown in FIG. 1 or an artificial vessel-vein anastomosis as shown in FIG. 2.

As shown in FIG. 1, the shunt construction 1 can be formed by anastomosing a vein 4 to a small incision in an artery 3 in an arm 2 and allowing blood to flow from the artery 3 to the vein 4. In this case, blood flows from the artery 3 through an anastomosis site 6 to the vein 4 in the direction indicated by an arrow B.

Alternatively, as shown in FIG. 2, the shunt construction 1 can be formed by anastomosing one end of an artificial vessel 5 to a small incision in an artery 3 in an arm 2 and the other end of the artificial vessel 5 to a vein 4. In this case, blood flows from the artery 3 to the artificial vessel 5 and further to the vein 4 through an anastomosis site 6 in the direction indicated by an arrow B.

A blood vessel cover 10 in accordance with embodiments of the present invention can be placed on the periphery of the vein 4 from the anastomosis site 6 to downstream in either the arteriovenous anastomosis shown in FIG. 1 or the artificial vessel-vein anastomosis shown in FIG. 2, and can remodel the vein 4 into a buffer system vessel.

In all of the above cases, the blood vessel cover 10 is preferably placed from the most upstream side of the vein 4 at the anastomosis site 6. The blood vessel cover 10 has a first end 10a and a second end 10b, the first end 10a is preferably placed at the most upstream side of the vein at the anastomosis site 6, and the second end 10b is preferably placed downstream of the vein 4 away from the anastomosis site 6.

The blood vessel cover 10 is formed in a continuous cylindrical shape over its entire circumference and has an axial direction x and a radial direction y. The axial direction x of the blood vessel cover 10 is the direction in which a central axis C of the blood vessel cover 10 extends, and the radial direction y of the blood vessel cover 10 is the direction connecting the central axis C of the blood vessel cover 10 and a point on the outer edge of the blood vessel cover 10 in a cross-section perpendicular to the axial direction x. The blood vessel cover 10 may be a knitted fabric, woven fabric, or net that is continuous around its entire circumference. Although the knitted fabric, woven fabric, and net have knitted stitches or weaves, gaps formed by those knitted stitches and weaves are not discontinuous portions of the blood vessel cover 10, and the knitted fabric, woven fabric, or net can form the above-described “continuous cylindrical shape.”

The blood vessel cover 10 is preferably flexible, and the axial direction x of the blood vessel cover 10 is preferably able to follow and curve with the direction of extension of the vein 4 to be covered.

As shown in FIG. 3, the blood vessel cover 10 preferably has a circular or oval lumen in shape in the cross-section in the radial direction y. Depending on the material constituting the blood vessel cover 10 and the structure of the blood vessel cover 10, the outer edge of the shape in the cross-section in the radial direction y may have fine irregularities.

Alternatively, although not shown in the figures, the blood vessel cover 10 may have a collapsed lumen under its own weight in its natural state. In such a case, the lumen can be widened to define the same axial direction x, radial direction y, and shape in a cross-section in the radial direction y as described above. The method of widening the lumen collapsed by its own weight includes, for example, a method where a tube whose lumen does not collapse under its own weight and which has a central axis parallel to the central axis C of the blood vessel cover 10 and is inscribed on the inner wall of the blood vessel cover 10 is inserted into the lumen of the blood vessel cover 10.

As shown in FIG. 1 to FIG. 5, the blood vessel cover 10 is a cylindrical cover placed on an outer circumference of the vein 4 that is anastomosed to the artery 3 or to the artificial vessel 5, has the first end 10a and the second end 10b in the axial direction x of the blood vessel cover 10, and the blood vessel cover 10 has a first part 11 from the first end 10a to a midpoint 10c between the first end 10a and the second end 10b and a second part 12 from the midpoint 10c to the second end 10b, wherein a diameter d2 of a second virtual cylinder T2 having a central axis parallel to the central axis C of the blood vessel cover 10 and inscribed in an inner wall of the second part 12 is larger than a diameter d1 of a first virtual cylinder T1 having a central axis parallel to the central axis C of the blood vessel cover 10 and inscribed in an inner wall of the first part 11.

With the above configuration, the minimum inner diameter of the second part 12 can be larger than the minimum inner diameter of the first part 11 of the blood vessel cover 10. By placing the first part 11 of the blood vessel cover 10 on the upstream side of the anastomosed vein 4 at the anastomosis site 6 and the second part 12 on the downstream side, the downstream side of the vein 4 can be covered more loosely than the upstream side. This allows the vein 4 to be remodeled so that it becomes a buffer system vessel where the pulsatile blood flow with high arterial pressure that flows into the vein 4 can be gradually buffered towards downstream and finally shifted to venous blood flow. As a result, blood turbulence and pulsatile changes in the venous wall are suppressed, preventing lesions such as intimal thickening.

More specifically, by gradually forming a two-layer structure, in the wall of the vein 4 at the shunt construction 1, comprising a smooth muscle layer containing elastic fiber layer thicker than the smooth muscle layer of normal veins and an elastic fiber layer containing collagen fibers thicker than the smooth muscle layer on the outside of the smooth muscle layer, the vein 4 at the shunt construction 1 can be remodeled into a buffer system vessel.

The difference between the above-described buffer system vessel and normal arteries is explained below: Blood vessels are composed of three layers: intima, tunica media, and adventitia. Of these, the intima contributes significantly to the anti-coagulability, but its mechanical contribution is very small. The composition of the mechanical elements of the arteries of the extremities, which are normally used for dialysis, consists largely of the tunica media containing some elastic fibers and abundant smooth muscle and the adventitia consisting of elastic fibers and collagen fibers etc. outside the tunica media. In other words, these arteries have very abundant smooth muscle and relatively fewer elastic fibers (the composition of “smooth muscle>elastic fibers”). The elastic fibers, due to their elasticity, have a buffering function like a lubber tube, resisting and relaxing the high pulsatile arterial blood pressure. On the other hand, the smooth muscle, which is muscle, has a more active mechanical function, resisting arterial blood pressure, and at the same time, has the active and proactive function of delivering high pulsatile arterial blood pressure to the periphery without attenuation. Because of this pressure delivering function of the abundant smooth muscle of arteries, the blood pressure is almost the same whether it is a large artery with an inner diameter of centimeters or a small artery with an inner diameter of a fraction of millimeter. In other words, normal arteries (with the configuration of “smooth muscle>elastic fibers”) do not have the ability to buffer high pulsatile arterial pressure due to the function of the abundant smooth muscle. On the other hand, when a vein at the shunt construction is remodeled into a buffer system vessel, the ratio of elastic fibers and smooth muscle is opposite of that in normal arteries, with elastic fibers abundant and smooth muscle relatively thin (configuration of “elastic fibers>smooth muscle”). Accordingly, in buffer system vessels, the pressure buffering function is dominant over the pressure delivery function. In buffer system vessels, while maintaining the above configuration “elastic fibers>smooth muscle”, i.e., the buffering function, the whole system gradually becomes thin and transformed to veins, i.e., gradually transformed to the normal veins as the buffered pressure decreases. If, hypothetically, the vein wall changes to normal artery-like, this is arterialization (remodeling into an artery) and not remodeling into the buffer system vessel. If that arterialization gradually weakens and spontaneously transitions to completely normal veins at the downstream part, it means that the vessel maintaining the configuration of “smooth muscle>elastic fibers” gradually becomes thin and transformed to veins, causing pathological changes in the downstream veins since it has no buffering function and high pulsatile blood pressure acts on the venous wall. This is the clear functional difference between the gradual thinning of the buffer system vessels and their transition to normal veins and the gradual thinning of normal arterial-like vessels and their transition to normal veins.

The blood vessel cover 10 in accordance with embodiments of the present invention, having the above configuration, can loosely cover the downstream side of the vein 4 at the shunt construction 1, allowing the wall structure of the vein 4 to be changed as described above and remodeled into a buffer system vessel.

Furthermore, while the vein 4 may gradually grow outward in the process of remodeling into a buffer system vessel, the blood vessel cover 10 in accordance with embodiments of the present invention can cover the downstream side of the vein 4 more loosely, thus reducing the inhibition by the blood vessel cover 10 to the growth of the vein 4 and allowing the lumen of the vein 4 to be kept wide to ensure sufficient blood flow.

This makes it possible to form the shunt construction 1 that can ensure sufficient blood flow while suppressing lesions such as intimal thickening, according to the blood vessel cover 10 in accordance with embodiments of the present invention.

Although not shown in the figures, the blood vessel cover 10 may be arranged to cover not only the vein 4 but also a part of the artery 3 and the artificial vessel 5 on the side of the anastomosis site 6 even when anastomosed to either the artery 3 or the artificial vessel 5.

The blood vessel cover 10 is cylindrical, and may have joints, for example, formed by rounding a flat-shaped material into a tubular shape and joining it by sutures or other methods. In such a case, the joints including sutures are preferably formed on the outer surface of the blood vessel cover 10. This prevents the joints from affecting the vein 4. Alternatively, a seamless tubular member without joints may be formed into the blood vessel cover 10 by using a molded or knitted member.

As shown in FIG. 3, the first virtual cylinder T1 is inscribed in the inner wall of the first part 11 at the first end 10a of the blood vessel cover 10, and parts other than the first end 10a may not be inscribed in the inner wall of the first part 11. In other words, the first part 11 of the blood vessel cover 10 have an inner diameter d1 at the first end 10a, and the parts other than the first end 10a may have an inner diameter larger than d1. In addition, as shown in FIG. 3, the second virtual cylinder T2 is inscribed in the inner wall of the second part 12 at the midpoint 10c of the blood vessel cover 10, and parts other than the midpoint 10c may not be inscribed in the inner wall of the second part 12. In other words, the second part 12 of the blood vessel cover 10 has an inner diameter d2 at the midpoint 10c, and the parts other than the midpoint 10c may have an inner diameter larger than d2. An example of such a shape of the blood vessel cover 10 includes a tapered shape in which the inner diameter increases progressively from the first end 10a to the second end 10b.

Even when the parts of the first part 11 other than the first end 10a have an inner diameter larger than d1, the inner diameter of the first part 11 is preferably the same as or smaller than the minimum inner diameter of the second part 12 over the entire axial direction x of the first part 11.

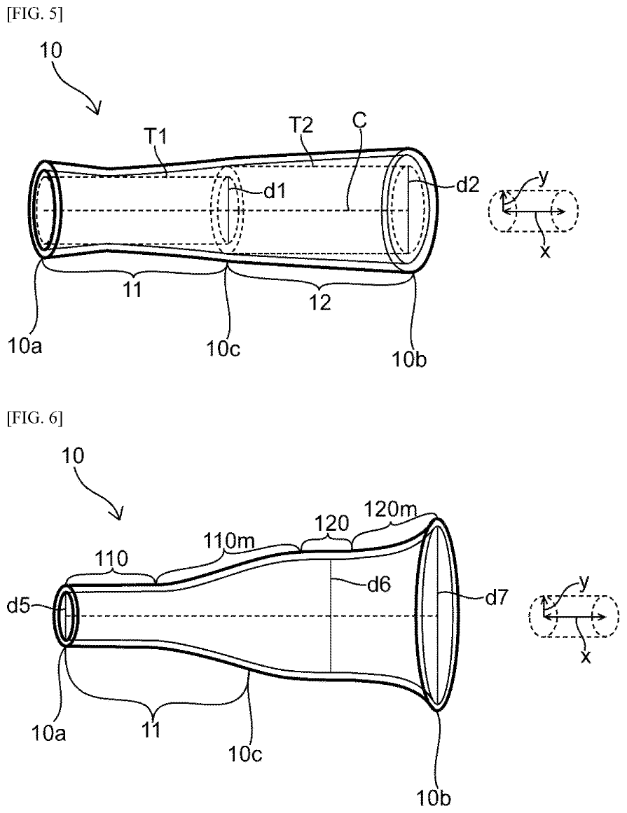

Alternatively, as shown in FIG. 4, the virtual cylinder T1 may be inscribed in the inner wall of the first part 11 in the axial direction x from the first end 10a of the blood vessel cover 10 to a predetermined position, and may not be inscribed in the inner wall of the first part 11 in portions other than the above portion including the first end 10a. In other words, the first part 11 of the blood vessel cover 10 has an inner diameter d1 at the portion from the first end 10a of the blood vessel cover 10 to a predetermined position, and may have an inner diameter larger than d1 at other portions.

With these shapes, when the first end 10a of the blood vessel cover 10 is placed on the upstream side of the vein 4 at the shunt construction 1, the part having the smallest inner diameter of the blood vessel cover 10 can cover the most upstream part of the vein 4 on the side of the anastomosis site 6, and the blood vessel cover 10 the inner diameter of which gradually increases as it is more downstream of the vein can cover the vein 4, allowing the vein 4 to be more loosely covered as it is more downstream, and thus facilitating remodeling of the vein 4 into a buffer system vessel and securing the lumen diameter of the vein 4.

Alternatively, as shown in FIG. 5, the first virtual cylinder T1 may be inscribed in the inner wall of the first part 11 at any position in the axial direction x between the first end 10a and the midpoint 10c of the blood vessel cover 10, and may not be inscribed in the inner wall of the first part 11 at any part other than the above position. In other words, the first part 11 of the blood vessel cover 10 has an inner diameter d1 at the position between the first end 10a and the midpoint 10c of the blood vessel cover 10, and may have an inner diameter larger than d1 at other portions.

In any of the above cases, the inner diameter of the first part 11 is preferably the same as or smaller than the minimum inner diameter of the second part 12 over the entire axial direction x of the first part 11.

Alternatively, although not shown in the figures, the first virtual cylinder T1 may be inscribed in the inner wall of the first part 11 over the entire section from the first end 10a to the midpoint 10c of the blood vessel cover 10 in the axial direction x. In other words, the first part 11 of the blood vessel cover 10 may have the inner diameter d1 over the entire axial direction x.

The second virtual cylinder T2 may be inscribed in the second part 12 at a part other than the midpoint 10c of the blood vessel cover 10. The second part 12 may have an inner diameter larger than d2 at a position other than the part where the second virtual cylinder T2 is inscribed. In such a case, the inner diameter of the second part 12 is preferably 2.5 times or less the diameter d2 of the second virtual cylinder T2, more preferably 2 times or less, and even more preferably 1.5 times or less over the entire axial direction x. The inner diameter of the second part 12 at the second end 10b is preferably larger than the inner diameter of the second part 12 at the midpoint 10c.

Alternatively, although not shown in the figures, the second virtual cylinder T2 may be inscribed in the inner wall of the second part 12 over the entire section from the midpoint 10c to the second end 10b of the blood vessel cover 10 in the axial direction x. In other words, the second part 12 of the blood vessel cover 10 may have the inner diameter of d2 throughout the entire axial direction x.

In any of the above embodiments, the diameter d2 of the second virtual cylinder T2 is larger than the diameter d1 of the first virtual cylinder T1, which allows the downstream of the vein 4 to be more loosely covered when the first end 10a of the blood vessel cover 10 is placed on the upstream side of the vein 4 at the shunt construction 1, achieving the above effects.

FIG. 6 show a perspective view of the blood vessel cover 10 in accordance with another embodiment. The blood vessel cover 10 of this embodiment preferably has a first end part 110 that is from the first end 10a to a midpoint of the first part 11 of the blood vessel cover 10 in the axial direction x of the blood vessel cover 10, and that has a first inner diameter d5; a middle part 120 that is located at the second end 10b side to the first end part 110, and that has a second inner diameter d6 of 1.2 times or more the first inner diameter d5: a first transition part 110m with a progressively increasing inner diameter between the first end part 110 and the middle part 120; and a second transition part 120m with a progressively increasing inner diameter between the middle part 120 and the second end 10b, wherein the second end 10b has a third diameter d7 of 1.2 times or more the second inner diameter d6.

Placing the first end part 110 of the blood vessel cover 10 having such a configuration on the upstream side of the vein 4 at the shunt construction 1 allows the vein 4 to spread slowly outward in the radial direction y as it is at more downstream side in the process of remodeling the vein 4 into a buffer system vessel, which facilitates the remodeling of the vein 4 into a buffer system vessel and makes it easier to maintain the lumen of the vein 4 to ensure blood flow. In addition, the second end 10b is the part where the restriction by the blood vessel cover 10 on the vein 4 is suddenly released, but the inner diameter d7 of this part has the above relationship to the middle part 120, which can mitigate the effect of the sudden release of the restriction by the blood vessel cover 10 on the vein 4.

In the case of the above configuration, in a cross-section in the axial direction x, a boundary between the first end part 110 and the first transition part 110m, a boundary between the first transition part 110m and the middle part 120, and a boundary between the middle part 120 and the second transition part 120m are preferably curved. Since the first end part 110, the middle part 120, and the second end 10b each have an inner diameter that differs by 1.2 times or more, the inner wall of the blood vessel cover 10 has a step in the axial direction. However, the step can be made smooth by having the respective boundaries as curved as described above. This allows the vessel to be covered with the blood vessel cover having a smooth lumen wall, which makes remodeling into a buffer system vessel easier.

A force required to expand the inner wall of the second part 12 by 1.5 times in the radial direction y from its natural state is preferably less than a force required to expand the inner wall of the first part 11 by 1.5 times in the radial direction y from its natural state.

The method of expanding the inner wall of the first part 11 by 1.5 times in the radial direction y from its natural state includes, for example, a method where a resin tube is inserted into the lumen of the blood vessel cover so that it is inscribed in the inner wall of the first part 11, and fluid is introduced into the lumen of the resin tube to apply pressure. In a similar manner, the inner wall of the second part 12 can be expanded by 1.5 times in the radial direction y from its natural state.

Alternatively, a cylindrical sample may be prepared by cutting out a portion of the first part 11 to have a predetermined length in the axial direction x, two pins may be inserted into the lumen of the cylindrical sample parallel to the axial direction of the cylindrical sample, and two pins may be pulled in opposite directions in the radial direction y from each other so that the inner diameter of the cylindrical sample is 1.5 times larger. In a similar manner, the inner wall of the second part 12 can be expanded by 1.5 times in the radial direction y from its natural state, but the length in the axial direction x of the cylindrical sample obtained by cutting out from the second part 12 should be the same as the length in the axial direction x of the cylindrical sample cut out from the first part 11.

The blood vessel cover 10 having the above configuration can more easily cover the downstream side of the vein 4 more loosely when the first part 11 of the blood vessel cover 10 is placed on the upstream side of the vein 4 at the shunt construction 1, effectively remodeling the vein 4 into a buffer system vessel and securing the lumen diameter of the vein 4.

The length of the blood vessel 10 in the axial direction x is preferably 5 mm or longer. The length of the blood vessel cover 10 in the axial direction x is more preferably 10 mm or longer, even more preferably 20 mm or longer, particularly preferably 30 mm or longer, and may be 40 mm or longer. The length of the blood vessel cover 10 in the axial direction x is preferably 120 mm or shorter, more preferably 100 mm or shorter, and even more preferably 90 mm or shorter. With the length of the blood vessel cover 10 in the axial direction x in the above range, the vein 4 at the shunt construction 1 can be covered with the blood vessel cover 10 longer than specified, facilitating remodeling of the vein 4 into a buffer system vessel.

The inner diameter of the blood vessel cover 10 is, at the portion with the minimum inner diameter in the first part 11, i.e., the portion inscribed by the first virtual cylinder T1, preferably 2 mm or more, more preferably 3 mm or more, even more preferably 4 mm or more, particularly preferably 5 mm or more, and preferably 10 mm or less, more preferably 8 mm or less, even more preferably 6 mm or less. The inner diameter of the blood vessel cover 10 is, at the portion with the minimum inner diameter in the second part 12, i.e., the portion inscribed by the second virtual cylinder T2, preferably 4 mm or more, more preferably 5 mm or more, even more preferably 6 mm or more, particularly preferably 7 mm or more, and preferably 12 mm or less, more preferably 10 mm or less, even more preferably 9 mm or less, particularly preferably 8 mm or less. Depending on the materials used to construct the blood vessel cover 10 and the structure of the blood vessel cover 10, the inner wall of the blood vessel cover 10 may have minor irregularities that make it difficult to determine the inner diameter, in which case the diameter d1 of the first virtual cylinder T1 inscribed in the first part 11 or the diameter d2 of the second virtual cylinder T2 inscribed in the second part 12 can be used as the inner diameter of the blood vessel cover 10.

The blood vessel cover 10 preferably has at least one of knitted fabric, woven fabric, and nonwoven fabric as a partial or whole component. With these materials, it is easy to form an elastic deformable blood vessel cover 10.

The type of knitted fabric is not limited and may be warp or weft knitting. Examples of knitting texture of the warp knitting include half knitting, back half knitting, quinz coat knitting, and satin knitting. The weft knitting includes circular knitting and flat knitting, and examples of knitting texture of the weft knitting include plain knitting, rib knitting, double side knitting, milan-rib knitting, and jacquard knitting. In view of excellence in elasticity, the knitted fabric is preferably composed of a weft knitted fabric. The type of woven fabric is not limited and may be plain weave, twill weave, vermillion weave, etc. Alternatively, the blood vessel cover 10 may composed of nonwoven fabric made by any method, such as melt-blown, needle-punched, spun-laced, electrospun, etc. The blood vessel cover 10 may be made of a combination of two or more different materials, for example, part of the cover is made of knitted fabric and the rest of the cover is made of another material, for example, nonwoven fabric.

The property of the wall of the shunt cover in which plastic deformation is predominant has a better buffering function than that in which elastic deformation is predominant. Therefore, the yarns forming knitted, woven, and nonwoven fabrics are also preferably composed of resin materials in which plastic deformation is predominant. For example, the yarns in which plastic deformation is predominant are exemplified by the ones made polyolefin-based resins such as polyethylene and polypropylene: polyamide-based resins such as nylon: polyester-based resins such as polyethylene terephthalate: polyimide-based resins: fluorine-based resins such as PTFE, PFA, and ETFE; and polyvinyl chloride-based resins. The yarns forming the knitted or woven fabric may be composed of resin materials used in artificial blood vessels (e.g., polyester, PTFE), and specifically exemplified by ePTFE, which is stretched PTFE, Dacron (registered trademark), which is polyester fibers made by Dupont. The blood vessel cover 10 may also be composed of biodegradable materials, such as aliphatic polyesters such as polylactic acid, polyglycolic acid, and polyhydroxyalkanoic acid; and aliphatic polyethers. In addition, the yarns forming the knitted or woven fabric may be composed of natural fibers, such as silk and cotton, or combination of resin materials, biodegradable materials, and natural fibers.

As shown in FIG. 7, preferably, the blood vessel cover 10 has a bellows structure with periodically repeating peaks and valleys in the axial direction x, wherein, in the axial direction x, the blood vessel cover 10 has the first end part 110 from the first end 10a to the midpoint of the first part 11, the distance L2 between adjacent peaks in the second part 12 is longer than the distance L1 between adjacent peaks in the first end part 110. The relatively shorter distance between the peaks of the bellows structure in the first end part 110 and the relatively longer distance in the second part 12 makes it easier to cover the downstream of the vein 4 more loosely when the first end part 110 is placed on the upstream side of the vein 4 at the shunt construction 1, making it easier to remodel the vein 4 into a buffer system vessel and to secure the lumen diameter of the vein 4.

Assessment of whether the vein 4 has been remodeled into a low-pressure buffer system vessel should be confirmed by both morphological confirmation and confirmation by measurement of the buffering effect, as described below. The morphological method can be performed by checking whether a two-layered structure is formed, consisting of a smooth muscle layer containing morphological elastic fibers and an outer layer of elastic fibers containing collagen fibers that are thicker than the smooth muscle layer. Specifically, the vein 4 at the shunt construction 1 is cut out, special stains such as hematoxylin-eosin (HE) stain and Elastica van Gieson (EvG) stain are applied to observe the vein wall cross section under a microscope. For example, with EvG staining, the smooth muscle is stained turbid yellow, the elastic fibers are stained dark purple, and the collagen fibers are stained dark led, so that the evaluation can be done by observing the smooth muscle layer containing elastic fibers and the elastic fiber layer containing collagen fibers to confirm “thickness of the smooth muscle layer containing elastic fibers<thickness of the elastic fiber layer containing collagen fibers.”

The method of confirming whether the vein 4 has been remodeled into a buffer system vessel by measuring the buffering effect can be performed by Doppler blood flow measurement and blood flow measurement with a color Doppler ultrasound imaging system.

The present application claims priority based on Japanese Patent Application No. 2021-146521 filed on Sep. 8, 2021. All the contents described in Japanese Patent Application No. 2021-146521 filed on Sep. 8, 2021 are incorporated herein by reference.

### DESCRIPTION OF REFERENCE SIGNS

1: shunt construction
2: arm
3: artery
4: vein
5: artificial blood vessel
6: anastomosis site
10: blood vessel cover
10a: first end of the blood vessel cover
10b: second end of the blood vessel cover
10c: midpoint of the blood vessel cover
11: first part
12: second part
110: first end part
110m: first transition part
120: middle part
120m: second transition part
C: central axis of the blood vessel cover
d1: diameter of the first virtual cylinder
d2: diameter of the second virtual cylinder
d5: inner diameter of the first end part
do: inner diameter of the middle part
d7: inner diameter of the second end
L1: distance between peaks in the first end part
L2: distance between peaks in the second part
T1: first virtual cylinder
T2: second virtual cylinder
x: axial direction
v: radial direction

1. A cylindrical blood vessel cover that is continuous around the entire circumference and to be placed on an outer circumference of a vein that is anastomosed to an artery or to an artificial vessel, comprising:
a first end and a second end in an axial direction of the blood vessel cover; and
a first part from the first end to a midpoint between the first end and the second end, and a second part from the midpoint to the second end, wherein
a diameter of a second virtual cylinder having a central axis parallel to a central axis of the blood vessel cover and inscribed in an inner wall of the second part is larger than a diameter of a first virtual cylinder having a central axis parallel to a central axis of the blood vessel cover and inscribed in an inner wall of the first part.

2. The blood vessel cover according to claim 1, comprising:
a first end part that is from the first end to a midpoint of the first part of the blood vessel cover in the axial direction, and that has a first inner diameter;
a middle part that is located on the second end side to the first end part, and that has a second inner diameter of 1.2 times or more the first inner diameter;
a first transition part with a progressively increasing inner diameter between the first end part and the middle part; and
a second transition part with a progressively increasing inner diameter between the middle part and the second end, wherein
the second end has a third inner diameter of 1.2 times or more the second inner diameter.

3. The blood vessel cover according to claim 2, wherein, in a cross-section in the axial direction, a boundary between the first end part and the first transition part, a boundary between the first transition part and the middle part, and a boundary between the middle part and the second transition part are curved.

4. The blood vessel cover according to claim 1, wherein a force required to expand the inner wall of the second part by 1.5 times in a radial direction from its natural state is less than a force required to expand the inner wall of the first part by 1.5 times in the radial direction from its natural state.

5. The blood vessel cover according to claim 1, having a length in the axial direction of 5 mm or longer.

6. The blood vessel cover according to claim 1, comprising at least one of knit fabric, woven fabric, and nonwoven fabric as a partial or whole component.

7. The blood vessel cover according to claim 1, having a bellows structure with periodically repeating peaks and valleys in the axial direction, wherein
in the axial direction, the blood vessel cover has a first end part from the first end to a midpoint of the first part, and a distance between adjacent peaks in the second part is longer than a distance between adjacent peaks in the first end part.
